(19) United States
(12) Patent Application Publication
(10) Pub. No.: US 2022/0064085 A1
(43) Pub. Date: Mar. 3, 2022

(54) LINEAR-MONOOLEFIN MANUFACTURING METHOD AND COMPOUND MANUFACTURING METHOD

(71) Applicants: ENEOS Corporation, Tokyo (JP); JSR CORPORATION, Tokyo (JP)

(72) Inventors: Sosuke HIGUCHI, Tokyo (JP); Yukihiro YOSHIWARA, Tokyo (JP); Nobuhiro KIMURA, Tokyo (JP); Mayu SUGIMOTO, Tokyo (JP); Junjie WANG, Tokyo (JP)

(73) Assignees: ENEOS Corporation, Tokyo (JP); JSR CORPORATION, Tokyo (JP)

(21) Appl. No.: 17/414,147

(22) PCT Filed: Dec. 18, 2019
(86) PCT No.: PCT/JP2019/049702

§ 371 Date: Jun. 15, 2021

(30) Foreign Application Priority Data
Dec. 18, 2018 (JP) 2018-236413

(51) Int. Cl. C07C 5/25; C07C 5/333; C07C 45/50

(52) U.S. Cl. CPC C07C 5/2518; C07C 45/50; C07C 5/333

(57) ABSTRACT

A method for producing linear monoolefins comprises a step of contacting a raw material composition containing a first linear monoolefin having 4 to 8 carbon atoms with an isomerization catalyst at 250 to 390° C. in the presence of 20 ppm by volume or more of molecular oxygen and/or 20 ppm by volume or more of water to perform an isomerization reaction for isomerizing at least a part of the first linear monoolefin to a second linear monoolefin having a different double bond position, wherein the catalyst contains zeolite.

### TECHNICAL FIELD

The present invention relates to a method for producing linear monoolefins. The present invention also relates to a method for producing compounds derived from linear monoolefins.

### BACKGROUND ART

Linear monoolefins having one double bond in a molecule are useful as basic chemical raw materials in the petrochemical industry, and their use depends on the position of the double bond in the molecule. Internal olefins having a double bond inside are used, for example, as reaction raw materials for hydrogenation, alkylation, etc. On the other hand, terminal olefins having a double bond at a terminal are used for reactions such as dehydrogenation, hydroformylation and oligomerization. Among the terminal olefins, C4 to C8 terminal olefins (e.g., 1-butene, 1-hexene and 1-octane) used as comonomers in the production of linear low density polyethylene (LLDPE) together with ethylene are particularly economically important. Further, 1-butene is also used for the production of butadiene, 1-polybutene, and butene oxide.

Linear olefins having a double bond at a terminal (e.g., 1-butene) may be produced, for example, by catalytically isomerizing linear olefins having a double bond inside (e.g., 2-butene).

For example, in Patent Literatures 1 to 5, a catalytic reaction for isomerizing linear olefins having an internal double bond to linear olefins having a terminal double bond is disclosed.

### CITATION LIST

### Patent Literature

Patent Literature 1: U.S. Pat. No. 3,642,933

Patent Literature 2: U.S. Pat. No. 4,229,610

Patent Literature 3: German Patent No. 3319171

Patent Literature 4: German Patent No. 3319099

Patent Literature 5: U.S. Pat. No. 4,289,919

### SUMMARY OF INVENTION

### Technical Problem

However, a position isomerization method of olefins using a conventional isomerization catalyst in an environment where oxygen or water is substantially present has drawbacks, such as decrease in purity of products due to the progress of side reactions, insufficient yield, and significant deterioration of the catalyst, causing difficulty in industrial use.

One of the objects of the present invention is to provide a method for producing linear monoolefins, capable of efficiently performing an isomerization reaction of olefins, with the catalyst deterioration sufficiently suppressed even in an environment where oxygen or water is substantially present. Another object of the present invention is to provide a method for producing compounds derived from linear monoolefins through a reaction of the linear monoolefins after isomerization.

### Solution to Problem

One aspect of the present invention relates to a method for producing linear monoolefins comprising a step of contacting a raw material composition containing a first linear monoolefin having 4 to 8 carbon atoms with an isomerization catalyst at 250 to 390° C. in the presence of 20 ppm by volume or more of molecular oxygen and/or 20 ppm by volume or more of water to perform an isomerization reaction for isomerizing at least a part of the first linear monoolefin to a second linear monoolefin having a different double bond position. Here, the catalyst contains zeolite.

In the production method according to an embodiment, the CO₂ selectivity in the isomerization reaction may be 0.001% or more and 0.09% or less.

In an embodiment, the first linear monoolefin and the second linear monoolefin may have 4 carbon atoms.

In an embodiment, the isomerization reaction may be a gas-solid catalytic reaction.

Another aspect of the present invention relates to a method for producing a compound, comprising a first step of contacting a first raw material composition containing a first linear monoolefin having 4 to 8 carbon atoms with an isomerization catalyst at 250 to 390° C. in the presence of 20 ppm by volume or more of molecular oxygen and/or 20 ppm by volume or more of water to perform an isomerization reaction for isomerizing at least a part of the first linear monoolefin to a second linear monoolefin having a different double bond position, and a second step of reacting a second raw material composition containing the second linear monoolefin to obtain a compound derived from the second linear monoolefin. Here, the catalyst contains zeolite.

In a production method according to an embodiment, the CO₂ selectivity in the isomerization reaction may be 0.001% or more and 0.09% or less.

In an embodiment, the second step may be a step of contacting the second raw material composition with a dehydrogenation catalyst to obtain a conjugated diene through a dehydrogenation reaction of the second linear monoolefin.

In one embodiment, the second step may be a step of contacting the second raw material composition with a hydroformylation catalyst to obtain an aldehyde through a hydroformylation reaction of the second linear monoolefin.

Still another aspect of the present invention relates to a method for producing a compound, comprising a step of contacting a raw material composition containing a first linear monoolefin having 4 to 8 carbon atoms with a catalyst group containing an isomerization catalyst at 250 to 390° C. in the presence of 20 ppm by volume or more of molecular oxygen and/or 20 ppm by volume or more of water to obtain a compound derived from the isomerized product of the first linear monoolefin. Here, the catalyst contains zeolite.

In an embodiment, the catalyst group may further include a dehydrogenation catalyst, and the compound may be a conjugated diene.

In an embodiment, the catalyst group may further comprise a hydroformylation catalyst, and the compound may be an aldehyde.

### Advantageous Effect of Invention

According to the present invention, a method for producing linear monoolefins, capable of efficiently performing an olefin isomerization reaction, with catalyst deterioration sufficiently suppressed even in an environment where oxygen or water is substantially present, is provided. Further, according to the present invention, a method for producing a compound comprising reacting a linear monoolefin after isomerization to obtain a compound derived from the linear monoolefin is provided.

### DESCRIPTION OF EMBODIMENTS

A preferred embodiment of the present invention is described below. The present invention, however, is not limited to the following embodiments.

A method for producing linear monoolefins according to the present embodiment comprises a step of contacting a raw material composition containing a first linear monoolefin having 4 to 8 carbon atoms with an isomerization catalyst at 250 to 390° C. in the presence of 20 ppm by volume or more of molecular oxygen (O₂) and/or 20 ppm by volume or more of water (steam) to perform an isomerization reaction for isomerizing at least a part of the first linear monoolefin to a second linear monoolefin having a different double bond position. In the present embodiment, the catalyst contains zeolite.

According to the production method according to the present embodiment, an isomerization reaction of olefins can be efficiently performed, with the catalyst deterioration sufficiently suppressed even in the case where 20 ppm by volume or more of molecular oxygen (hereinafter, also referred to simply as oxygen) and/or 20 ppm by volume or more of water are present.

Regarding isomerization of linear monoolefins, for example, isomerization of 2-butene to 1-butene is limited by the thermodynamic equilibrium of n-butene isomers. It is known that the maximum achievable concentration of 1-butene in n-butenes is about 22% at 400° C. and about 30% at 500° C. due to the thermodynamic equilibrium for a single pass in a reactor (for example, Japanese Unexamined Patent Publication No. H8-224470). In the method for producing linear monoolefins in the present embodiment, isomerization can be achieved at a level close to the theoretical value (for example, 70% or more of the theoretical value) even in the presence of oxygen and water, and the catalytic activity is maintained for a long period.

The method for producing linear monoolefins according to the present embodiment is performed in the presence of oxygen and/or water. The isomerization reaction using a conventional isomerization catalyst is usually performed in an environment where oxygen and water are absent (in particular, in absence of oxygen), and in the case where oxygen is present, many side reactions such as a complete oxidation reaction occur, so that allowing the isomerization of olefins to selectively proceed is difficult. On the other hand, in the method for producing linear monoolefins according to the present embodiment, the isomerization reaction of olefins can efficiently proceed with side reactions sufficiently suppressed, and the isomerization reaction can be performed for a long period due to excellent durability of the catalyst.

In the method for producing linear monoolefins according to the present embodiment, the isomerization reaction of olefins proceeds efficiently even in the presence of oxygen and/or water as described above, so that, for example, raw materials can be supplied without removing oxygen and water from the reaction in the previous stage, which is extremely advantageous for the process.

Also, the method for producing a linear monoolefins according to the present embodiment may be performed in parallel with another reaction that consumes the second linear olefin after isomerization. Here, since the isomerization catalyst for use in the production method according to the present embodiment allows the isomerization reaction to proceed efficiently even in the presence of oxygen and/or water, a reaction that proceeds in the presence of oxygen or water may be selected as the other reaction described above. For example, oxidative dehydrogenation reaction of olefins, hydroformylation reaction of olefins, or the like may be selected as the other reaction described above.

In the present embodiment, the isomerization catalyst and a catalyst for another reaction described above (e.g., dehydrogenation catalyst, hydroformylation catalyst) may be mixed to perform the isomerization reaction and another reaction at the same time. In this case, the second linear monoolefin is consumed by the other reaction, and the second linear monoolefin is produced by the isomerization reaction corresponding to the thermodynamic equilibrium, so that apparent reactivity of the isomerization reaction can be improved.

In the present embodiment, the first linear monoolefin may have 4 to 8 carbon atoms, or may have 4 carbon atoms.

The first linear monoolefin may be an internal olefin, or may be a terminal olefin.

The first linear monoolefin may be, for example, a linear monoolefin selected from the group consisting of 1-butene, trans-2-butene, cis-2-butene, 1-pentene, 2-pentene, 1-hexene, 2-hexene, 3-hexene, 1-octene, 2-octene, 3-octene and 4-octene. As the first linear monoolefin, one may be used alone, or two or more may be used in combination.

The first linear monoolefin may have a substituent containing a hetero atom such as oxygen, nitrogen, halogen and sulfur. Examples of the substituents may include at least one selected from the group consisting of a halogen atom (—F, —Cl, —Br, —I), a hydroxyl group (—OH), an alkoxy groups (—OR), a carboxyl group (—COOH), an ester group (—COOR), an aldehyde group (—CHO) and an acyl group (—C(═O)R). The raw materials containing the linear monoolefin having a substituent may be, for example, alcohols, ethers, or biofuels.

As the first linear monoolefin, it is not necessary to use an isolated linear monoolefin itself, and any form of mixture may be used on an as needed basis. In the case where the first linear monoolefin is butene, the raw material composition may be, for example, a C4 fraction obtained by fluid catalytic cracking of heavy oil fraction, or a C4 fraction obtained by thermal cracking of naphtha.

In the present embodiment, the raw material composition may contain other components other than the first linear monoolefin. The other component may be, for example, an isomerized product of the first linear monoolefin (which may include the second linear monoolefin), a saturated hydrocarbon compound, or a diene. The saturated hydrocarbon compound and the diene may be, for example, one having the same number of carbon atoms as the first linear monoolefin. The saturated hydrocarbon compound may be, for example, n-butane or cyclobutane. The diene may be, for example, butadiene. The raw material composition containing the first linear monoolefin may contain impurities such as hydrogen, nitrogen, carbon monoxide, carbon dioxide, and methane within a range where the effects of the present invention are not impaired. As the raw material composition, a raw material consisting of a linear monoolefin only may be used.

In the present embodiment, the concentration of the first linear monoolefin in the raw material composition is not particularly limited, and with increase in the concentration of the first linear monoolefin in the raw material composition, economic performance tends to improve.

The second linear monoolefin is an isomer having a double bond position different from that of the first linear monoolefin. Examples of the second linear monoolefin include the compounds shown as examples of the first linear monoolefin. The second linear monoolefin may be an internal olefin, or may be a terminal olefin.

In a suitable embodiment, for example, the first linear monoolefin may be an internal olefin, and the second linear monoolefin may be a terminal olefin. Further, the first linear monoolefin may be 2-butene, and the second linear monoolefin may be 1-butene.

The isomerization catalyst in the present embodiment is described in detail as follows.

The isomerization catalyst is a solid catalyst that catalyzes isomerization reaction of linear monoolefins (positional isomerization of olefins), including zeolite.

Crystalline aluminosilicates, which are collectively known as zeolite, have a micro space (nano space) of molecular size in one crystal. Further, there exist many types of zeolites classified according to the crystal structure thereof, including LTA (A type), MFI (ZSM-5 type), MOR, BEA, FER, FAU (X type, Y type), SAPO, and ALPO. The isomerization catalyst may contain any one of these zeolites, and may contain two or more of zeolites.

In the isomerization catalyst, the molar ratio of Si to Al (Si/Al) may be 5 or more, may be 100 or more, or may be 200 or more. Further, the molar ratio (Si/Al) may be 10000 or less, may be 3000 or less, or may be 2000 or less. The isomerization catalyst having such a ratio tends to suppress the catalyst deterioration more significantly in the presence of oxygen and/or water.

The isomerization catalyst may be a metal element supported on zeolite. The metal element to be supported (hereinafter, also referred to as a supported metal element) is not particularly limited, and may be, for example, an alkali metal, an alkaline earth metal, or a transition metal.

The supporting method of the supported metal element is not particularly limited, and may be, for example, an impregnation method, a deposition method, a coprecipitation method, a kneading method, an ion exchange method, or a pore filling method.

The supply source of the supported metal element may be, for example, at least one selected from the group consisting of an oxide, a nitrate, a carbonate, an ammonium salt, a hydroxide, a carboxylate, an ammonium carboxylate, an ammonium halide salt, a hydrogen acid (e.g., chloroplatinic acid (H₂PtCl₆)), an acetylacetonate and an alkoxide.

The content of the supported metal element in the isomerization catalyst is not particularly limited, and may be, for example, 0.01 to 100 parts by mass, or 0.1 to 50 parts by mass based on 100 parts by mass of inorganic oxides. The content of the supported metal element may be determined by the inductively coupled plasma emission spectroscopy (ICP emission spectroscopy).

As an effective method for characterizing the acidity of a catalyst, the ammonia temperature programmed desorption method (ammonia TPD, NH₃-TPD) is widely known. For example, C. V. Hidalgo et al., Journal of Catalysis, Vol. 85, pp. 362-369 (1984) have shown that the amount of Bronsted acid sites and the distribution of acid strength of Bronsted acid sites can be measured by the ammonia TPD method.

In the ammonia TPD method, ammonia as a base probe molecule is adsorbed on a sample solid, and the temperature is continuously raised to simultaneously measure the amount of desorbed ammonia and the temperature. Ammonia adsorbed on weak acid sites is desorbed at low temperature (corresponding to desorption in the low adsorption heat range), and ammonia adsorbed at strong acid sites is desorbed at high temperature (corresponding to desorption in the high adsorption heat range). In such an ammonia TPD method, the acid strength is indicated by the temperature and the heat of adsorption without use of coloring reaction, so the strength of solid acid and the amount of solid acid are represented by more accurate values to appropriately perform evaluation of the characteristics of an isomerization catalyst.

The amount of acid sites (acid amount) of an isomerization catalyst may be determined by the ammonia TPD method, in which the amount of ammonia adsorbed is measured with a device and measurement conditions described in “Niwa; Zeolite, 10, 175 (1993)”.

The amount of total acid sites (total acid amount) A₁ of the isomerization catalyst may be 0.11 mmol/g or less, may be 0.09 mmol/g or less, may be 0.03 mmol/g or less, may be 0.015 mmol/g or less, or may be 0.010 mmol/g or less. With a total acid amount in the range, skeletal isomerization, side reactions such as CO₂ generation, catalyst deterioration due to coke precipitation, etc., tend to be suppressed. Further, the total acid amount A₁ of the isomerization catalyst may be 0.001 mmol/g or more, or may be 0.003 mmol/g or more.

In the isomerization catalyst, the ratio of the amount of acid sites A₂ measured in the temperature range of 600° C. or more relative to the total amount of acid sites A₁, i.e., A₂/A₁, may be 0.03 or more, may be 0.05 or more, may be 0.08 or more, may be 0.1 or more, or may be 0.15 or more. With a ratio A₂/A₁ in the range, skeletal isomerization, side reactions such as CO₂ production, and catalyst deterioration due to coke precipitation tend to be suppressed. Further, the ratio A₂/A₁ may be 1 or less, or may be 0.7 or less.

The isomerization catalyst may be fired on an as needed basis. The firing may be performed in one stage or in multiple stages including two or more stages. The firing temperature is not particularly limited. In the case where the firing is performed in one stage, the firing temperature may be, for example, 200 to 600° C. The firing time may be 1 to 10 hours. Although the firing may be usually performed under air flow, the atmosphere during firing is not particularly limited.

From the viewpoint of improving moldability, the isomerization catalyst may contain a molding aid within a range where the physical properties and catalytic performance of the catalyst are not impaired. The molding aid may be, for example, at least one selected from the group consisting of thickeners, surfactants, water retention agents, plasticizers, and binder raw materials.

The isomerization catalyst may be molded by a method such as an extrusion molding method and a tablet molding method. The molding step may be performed at an appropriate stage of the production step of the isomerization catalyst in consideration of reactivity of the molding aid, etc.

The shape of the isomerization catalyst is not particularly limited and can be appropriately selected depending on the embodiment of using the catalyst. For example, the shape of the isomerization catalyst may be a shape of pellet, granule, honeycomb, sponge, or the like.

The isomerization reaction and other reactions in the present embodiment are described in detail as follows.

In the present embodiment, a raw material composition containing a first linear monoolefin is contacted with an isomerization catalyst at 250 to 390° C. in the presence of 20 ppm by volume or more of oxygen and/or 20 ppm by volume or more of water (steam) to perform an isomerization reaction of the first linear monoolefin. Through the isomerization reaction, at least a part of the first linear monoolefin is isomerized to a second linear monoolefin.

The temperature at which the raw material composition is contacted with the isomerization catalyst may also be referred to as the reaction temperature of the isomerization reaction.

The reaction temperature of the isomerization reaction is preferably 250 to 390° C., more preferably 300 to 390° C., still more preferably 320 to 390° C.

The amount of oxygen in the reaction system may be 20 ppm by volume or more, may be 0.01 vol % or more, may be 0.1 vol % or more, or may be 0.5 vol % or more. Further, the amount of oxygen may be 50 vol % or less, may be 30 vol % or less, or may be 20 vol % or less.

The amount of water in the reaction system may be 20 ppm by volume or more, may be 0.01 vol % or more, may be 0.1 vol % or more, or may be 0.5 vol % or more. Further, the amount of water may be 50 vol % or less, may be 30 vol % or less, or may be 20 vol % or less.

The isomerization reaction may be performed in an environment where other components other than the raw material composition, oxygen and water are further present. The other components may be, for example, methane, hydrogen, nitrogen, carbon dioxide, carbon monoxide, etc.

The isomerization reaction may be a gas-solid catalytic reaction or a liquid-solid catalytic reaction, and a gas-solid catalytic reaction is preferred. Incidentally, the gas-solid catalytic reaction refers to a reaction that is performed by contacting a gas-phase raw material with a solid-phase isomerization catalyst, and the liquid-solid catalytic reaction refers to a reaction that is performed by contacting a liquid-phase raw material with a solid-phase isomerization catalyst.

The isomerization reaction may be performed, for example, by feeding the raw material composition through a reactor filled with an isomerization catalyst.

In the isomerization reaction, oxygen and water that are present in the reaction system may be those supplied to a reactor together with the raw material composition. In other words, the isomerization reaction may be performed by feeding a raw material gas containing a raw material composition containing the first linear monoolefin, and 20 ppm by volume or more of oxygen and/or 20 ppm by volume or more of water, through a reactor filled with the isomerization catalyst.

The amount of oxygen in the raw material gas may be 20 ppm by volume or more, may be 0.01 vol % or more, may be 0.1 vol % or more, or may be 0.5 vol % or more. Further, the amount of oxygen in the raw material gas may be 50 vol % or less, may be 30 vol % or less, or may be 20 vol % or less.

The amount of water in the raw material gas may be 20 ppm by volume or more, may be 0.01 vol % or more, may be 0.1 vol % or more, or may be 0.5 vol % or more. Further, the amount of water in the raw material gas may be 50 vol % or less, may be 30 vol % or less, or may be 20 vol % or less.

The raw material gas may contain any impurities within a range where the effects of the invention are not impaired. Such impurities may be, for example, nitrogen, argon, neon, helium, carbon monoxide, or carbon dioxide.

In the present embodiment, it is preferable that the CO₂ selectivity in the isomerization reaction be 0.001% or more. Through adjustment of the reaction conditions of the isomerization reaction to have such a CO₂ selectivity, suppression of the catalyst deterioration and improvement in the isomerization efficiency are achieved. From the viewpoint of improving the reaction yield, the CO₂ selectivity is preferably 0.09% or less, more preferably 0.07% or less. The CO₂ selectivity in the isomerization reaction may be calculated by the following formula from the analysis result of the reaction product by gas chromatography. The amount of CO₂ and the total amount of each component are calculated by multiplying the peak area of each component measured by gas chromatography by a factor obtained from the calibration curve.

CO₂ selectivity=Amount of CO₂ (mol)/Total amount of each component in reaction product (mol)

In the present embodiment, the second linear monoolefin produced in the isomerization reaction may be subjected to another reaction to produce a compound derived from the second linear monoolefin.

In other words, the method for producing a compound of the present embodiment may comprise a first step of contacting a first raw material compound containing a first linear monoolefin with an isomerization catalyst in the presence of 20 ppm by volume or more of molecular oxygen and/or 20 ppm by volume or more of water to isomerize at least a part of the first linear monoolefin into a second linear monoolefin having a different double bond position, and a second step of reacting a second raw material composition containing the second linear monoolefin to obtain a compound derived from the second linear monoolefin.

The first step may be performed according to the preferred embodiment of the isomerization reaction described above. Various reactions for reacting the second linear monoolefin may be applied to the second step, and known reaction conditions may be applied to the reaction conditions.

The second step may be performed, for example, by feeding a raw material gas containing the second raw material composition through a reactor filled with a reaction catalyst.

In the second step, a produced gas after the isomerization reaction in the first step may be used as the second raw material composition. For example, the first step may be a step of feeding a raw material gas containing the first raw material composition through a first reactor filled with an isomerization catalyst to obtain a produced gas containing a second linear monoolefin, and the second step may be a step of feeding the produced gas obtained in the first step through a second reactor filled with a reaction catalyst to cause a reaction of the second linear monoolefin.

The second step may be a step of obtaining a target compound derived from the second linear monoolefin and a composition containing the first linear monoolefin. The first linear monoolefin in the composition may be, for example, the first linear monoolefin contained in the second raw material composition (for example, the produced gas in the first step) provided for use in the second step, or may be the first linear monoolefin produced in the reaction in the second step.

In the case where a composition containing the first linear monoolefin is obtained in the second step, the composition may be reused as a part or the whole of the first raw material composition in the first step. In the first step, the olefin isomerization reaction proceeds efficiently even in the presence of oxygen and water, so that oxygen and water are not required to be removed for the reuse, resulting in excellent efficiency of the entire process.

The second step may be a step of producing a conjugated diene by the oxidative dehydrogenation reaction of the second linear monoolefin. On this occasion, the second step may be a step of contacting the second raw material composition with a dehydrogenation catalyst to obtain a conjugated diene.

The reaction conditions for the oxidative dehydrogenation reaction are not particularly limited, and various known reaction conditions may be applied. For example, reaction conditions may be at 400° C. and 0.1 MPaG.

As the dehydrogenation catalyst, a known catalyst for dehydrogenation reaction may be used. Examples of the dehydrogenation catalyst include a multi-component molybdenum-bismuth catalyst, a ferrite catalyst, a vanadium-magnesium catalyst and a cobalt-molybdenum catalyst.

The second step may be a step of producing an aldehyde by a hydroformylation reaction of the second linear monoolefin. On this occasion, the second step may be a step of contacting the second raw material composition with a hydroformylation catalyst to obtain the aldehyde.

The reaction conditions for the hydroformylation reaction are not particularly limited, and various known reaction conditions may be applied. For example, the reaction conditions may be at 150° C. and 1.5 MPa.

As the hydroformylation catalyst, a known catalyst for hydroformylation reaction may be used. Examples of the hydroformylation catalyst include a rhodium catalyst and a cobalt catalyst.

In the present embodiment, another reaction that consumes the second linear monoolefin after isomerization may be performed simultaneously with the isomerization reaction.

The isomerization catalyst allows the isomerization reaction of olefins to proceed efficiently even in the presence of oxygen and/or water, so that a reaction that proceeds in the presence of oxygen or water may be selected as the other reaction described above. For example, oxidative dehydrogenation reaction of olefins, hydroformylation reaction of olefins, etc., may be selected as the other reactions described above.

In the present embodiment, the isomerization catalyst and a catalyst for the other reaction described above (for example, a dehydrogenation catalyst and a hydroformylation catalyst) may be mixed to perform the isomerization reaction and the other reaction at the same time. In this case, the second linear monoolefin is consumed by the other reaction, and the second linear monoolefin is produced by the isomerization reaction corresponding to the thermodynamic equilibrium, so that the apparent reactivity of the isomerization reaction can be improved.

In other words, the method for producing a compound of the present embodiment may comprise a step of contacting the raw material composition containing the first linear monoolefin with a catalyst group containing an isomerization catalyst in the presence of 20 ppm by volume or more of molecular oxygen and/or 20 ppm by volume or more of water to obtain a compound derived from the isomerized product of the first linear monoolefin. The isomerized product of the first linear monoolefin may be the second linear monoolefin described above.

Corresponding to an intended reaction, the catalyst group includes a catalyst other than the isomerization catalyst. For example, the other reaction described above may be an oxidative dehydrogenation reaction, and, in that case, the catalyst group may include an isomerization catalyst and a dehydrogenation catalyst. The other reaction described above may be a hydroformylation reaction, and, in that case, the catalyst group may include an isomerization catalyst and a hydroformylation catalyst. Examples of the dehydrogenation catalyst and the hydroformylation catalyst may include the same ones as described above.

In this embodiment, the step may be performed by feeding a raw material gas containing the raw material composition through a reactor filled with the catalyst group.

The step described above may be a step of obtaining a target compound derived from an isomerized product of the first linear monoolefin, and an unreacted material containing the first linear monoolefin. On this occasion, the unreacted material may be reused as a part or the whole of the raw material composition in the step described above. In the step described above, the olefin isomerization reaction proceeds efficiently even in the presence of oxygen and water, so that oxygen and water are not required to be removed for the reuse, resulting in excellent efficiency of the entire process.

Although the preferred embodiments of the present invention have been described above, the present invention is not limited thereto.

### EXAMPLES

The present invention is described in more detail with reference to Examples shown below, though the present invention is not limited thereto.

### Example 1

A reaction tube made of stainless steel with an inner diameter of 10.9 mm and a length of 300 mm was filled with 1.5 g of H-type-ZSM-5 zeolite catalyst (manufactured by Tosoh Corporation, SiO₂/Al₂O₃=1900 (mol/mol)). A raw material having a composition shown in Table 1 was subjected to mixing so as to have a ratio of linear butene:nitrogen:oxygen:steam=1:13.5:1.5:1.2 in the raw material gas, and supplied to a reactor heated to 250° C. in advance at a mass space velocity (WHSV (h⁻¹)) of linear butene in the raw material gas with respect to the catalyst became 2.7 h⁻¹ to perform an isomerization reaction.

TABLE 1

| Gas composition | (Vol %) |
|---|---|
| Butane | 32.2 |
| Cis-2-butene | 27.3 |
| Trans-2-butene | 40.4 |
| Isobutene | 0.0 |
| 1-butene | 0.1 |
| Total | 100.0 |

The production rate of 1-butene, the rate of reaching equilibrium, and the CO₂ selectivity at 1 hour after initiation of the reaction were as shown in Table 2. The production rate of 1-butene indicates the proportion of 1-butene in linear butenes in the produced gas. The rate of reaching equilibrium indicates the rate of reaching relative to the theoretical value (17.3%) for the rate of 1-butene through thermodynamic equilibrium at a reaction temperature (250° C.).

### Example 2

An isomerization reaction was performed in the same manner as in Example 1, except that the reaction temperature in the isomerization reaction was changed to 300° C. As a result, the production rate of 1-butene, the rate of reaching equilibrium, and the CO₂ selectivity at 1 hour after initiation of the reaction were as shown in Table 2. The rate of reaching equilibrium indicates the rate of reaching relative to the theoretical value (20.2%) for the rate of 1-butene through thermodynamic equilibrium at a reaction temperature (300° C.).

### Example 3

An isomerization reaction was performed in the same manner as in Example 1, except that the reaction temperature in the isomerization reaction was changed to 320° C., and WHSV was changed to 5.3 h⁻¹. As a result, the production rate of 1-butene, the rate of reaching equilibrium, the change rate of rate of reaching equilibrium, and the CO₂ selectivity at 1 hour after and 300 hours after initiation of the reaction were as shown in Table 2 and Table 3. The rate of reaching equilibrium indicates the rate of reaching relative to the theoretical value (21.2%) for the rate of 1-butene through thermodynamic equilibrium at a reaction temperature (320° C.).

### Example 4

An isomerization reaction was performed in the same manner as in Example 1, except that the reaction temperature in the isomerization reaction was changed to 350° C., and WHSV was changed to 10.6 h⁻¹. As a result, the production rate of 1-butene, the rate of reaching equilibrium, the change rate of rate of reaching equilibrium, and the CO₂ selectivity at 1 hour after and 300 hours after initiation of the reaction were as shown in Table 2 and Table 3. The rate of reaching equilibrium indicates the rate of reaching relative to the theoretical value (22.9%) for the rate of 1-butene through thermodynamic equilibrium at a reaction temperature (350° C.).

### Example 5

An isomerization reaction was performed in the same manner as in Example 1, except that the reaction temperature in the isomerization reaction was changed to 380° C., and WHSV was changed to 10.6 h⁻¹. As a result, the production rate of 1-butene, the rate of reaching equilibrium, the change rate of rate of reaching equilibrium, and the CO₂ selectivity at 1 hour after and 300 hours after initiation of the reaction were as shown in Table 2 and Table 3. The rate of reaching equilibrium indicates the rate of reaching relative to the theoretical value (24.0%) for the rate of 1-butene through thermodynamic equilibrium at a reaction temperature (380° C.).

### Comparative Example 1

An isomerization reaction was performed in the same manner as in Example 1, except that the reaction temperature in the isomerization reaction was changed to 230° C. As a result, the production rate of 1-butene, the rate of reaching equilibrium, and the CO₂ selectivity at 1 hour after initiation of the reaction were as shown in Table 2. The rate of reaching equilibrium indicates the rate of reaching relative to the theoretical value (15.5%) for the rate of 1-butene through thermodynamic equilibrium at a reaction temperature (230° C.).

### Comparative Example 2

An isomerization reaction was performed in the same manner as in Example 1, except that the reaction temperature in the isomerization reaction was changed to 400° C., and WHSV was changed to 10.6 h⁻¹. As a result, the production rate of 1-butene, the rate of reaching equilibrium, and the CO₂ selectivity at 1 hour after initiation of the reaction were as shown in Table 2. The rate of reaching equilibrium indicates the rate of reaching relative to the theoretical value (25.3%) for the rate of 1-butene through thermodynamic equilibrium at a reaction temperature (400° C.).

TABLE 2

| | | Comparative | Example | Example | Example | Example | Example | Comparative |
|---|---|---|---|---|---|---|---|---|
| | Unit | Example 1 | 1 | 2 | 3 | 4 | 5 | Example 2 |
| Reaction | ° C. | 230 | 250 | 300 | 320 | 350 | 380 | 400 |
| temperature | | | | | | | | |
| Reaction time | Hour | 1 | 1 | 1 | 1 | 1 | 1 | 1 |
| WHSV | h⁻¹ | 2.7 | 2.7 | 2.7 | 5.3 | 10.6 | 10.6 | 10.6 |
| Production rate of | % | 5.0 | 8.3 | 12 | 16.3 | 18.3 | 19.8 | 21.4 |
| 1 butene | | | | | | | | |
| Rate of reaching | % | 32.2 | 48 | 59.5 | 77.1 | 80 | 82.5 | 84.5 |
| equilibrium | | | | | | | | |
| CO2 selectivity | % | 0.008 | 0.008 | 0.009 | 0.01 | 0.02 | 0.05 | 0.1 |

TABLE 3

| | Unit | Example 3 | Example 4 | Example 5 |
|---|---|---|---|---|
| Reaction temperature | ° C. | 320 | 350 | 380 |
| Reaction time | Hour | 300 | 300 | 300 |
| WHSV | h⁻¹ | 5.3 | 10.6 | 10.6 |
| Production rate of | % | 15.6 | 18.1 | 19.7 |
| 1 butene | | | | |
| Rate of reaching | % | 73.4 | 79 | 82.1 |
| equilibrium | | | | |
| Change rate | % | −4.8 | −1 | −0.55 |
| CO₂ selectivity | % | 0.02 | 0.02 | 0.05 |

1. A method for producing linear monoolefins comprising:
contacting a raw material composition containing a first linear monoolefin having 4 to 8 carbon atoms with an isomerization catalyst at 250 to 390° C. in the presence of 20 ppm by volume or more of molecular oxygen and/or 20 ppm by volume or more of water to perform an isomerization reaction for isomerizing at least a part of the first linear monoolefin to a second linear monoolefin having a different double bond position,
wherein the catalyst contains zeolite.

2. The method for producing linear monoolefins according to claim 1,
wherein a CO₂ selectivity in the isomerization reaction is 0.001% or more and 0.09% or less.

3. The method for producing linear monoolefins according to claim 1,
wherein the first linear monoolefin and the second linear monoolefin have 4 carbon atoms.

4. The method for producing linear monoolefins according to claim 1,
wherein the isomerization reaction is a gas-solid catalytic reaction.

5. A method for producing a compound comprising:
contacting a first raw material composition containing a first linear monoolefin having 4 to 8 carbon atoms with an isomerization catalyst at 250 to 390° C. in the presence of 20 ppm by volume or more of molecular oxygen and/or 20 ppm by volume or more of water to perform an isomerization reaction for isomerizing at least a part of the first linear monoolefin to a second linear monoolefin having a different double bond position, and
reacting a second raw material composition containing the second linear monoolefin to obtain a compound derived from the second linear monoolefin,
wherein the catalyst contains zeolite.

6. The method for producing a compound according to claim 5,
wherein the CO2 selectivity in the isomerization reaction is 0.001% or more and 0.09% or less.

7. The method for producing a compound according to claim 5,
wherein the reacting a second raw material composition includes contacting the second raw material composition with a dehydrogenation catalyst to obtain a conjugated diene through a dehydrogenation reaction of the second linear monoolefin.

8. The method for producing a compound according to claim 5,
wherein the reacting a second raw material composition includes contacting the second raw material composition with a hydroformylation catalyst to obtain an aldehyde through a hydroformylation reaction of the second linear monoolefin.

9. A method for producing a compound comprising:
contacting a raw material composition containing a first linear monoolefin having 4 to 8 carbon atoms with a catalyst group containing an isomerization catalyst at 250 to 390° C. in the presence of 20 ppm by volume or more of molecular oxygen and/or 20 ppm by volume or more of water to obtain a compound derived from the isomerized product of the first linear monoolefin,
wherein the catalyst contains zeolite.

10. The method for producing a compound according to claim 9,
wherein the catalyst group further comprises a dehydrogenation catalyst, and
the compound is a conjugated diene.

11. The method for producing a compound according to claim 9,
wherein the catalyst group further comprises a hydroformylation catalyst, and
the compound is an aldehyde.
